(19) United States
(12) Patent Application Publication
(10) Pub. No.: US 2006/0069260 A1
(43) Pub. Date: Mar. 30, 2006

(54) Preparation of N-aryl pyridones

(76) Inventors: Huiping Zhang, Belle Mead, NJ (US); Bang-Chi Chen, Plainsboro, NJ (US); Rulin Zhao, Pennington, NJ (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(21) Appl. No.: 11/235,731

(22) Filed: Sep. 26, 2005

Related U.S. Application Data
(60) Provisional application No. 60/613,982, filed on Sep. 28, 2004.

(51) Int. Cl. C07D 213/70

(52) U.S. Cl. 546/290

(57) ABSTRACT

A novel process and intermediates thereof for making N-aryl pyridones of the type shown below from appropriate pyridinolates is described. 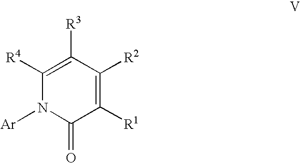 These compounds are useful as intermediates for the synthesis of clinical candidates.

### CROSS-REFERENCE TO RELATED APPLICATIONS

This application claims a benefit of a priority from U.S. Provisional Application No. 60/613,982 filed Sep. 28, 2004, the entire disclosure of which is herein incorporated by reference.

### FIELD OF THE INVENTION

The present invention relates generally to processes for the preparation of N-aryl pyridones, derivatives thereof, and intermediates for the synthesis of the same; such pyridones and derivatives being useful as intermediates for clinical candidates.

### BACKGROUND OF THE INVENTION

Compounds like those described in WO 03/26652 are currently being studied as factor Xa inhibitors in clinical settings. Clinical trials and NDA submissions require practical, large-scale synthesis of the active drug. Consequently, it is desirable to find new synthetic procedures for making intermediates that are useful in preparing compounds like those in WO 03/26652.

### SUMMARY OF THE INVENTION

Accordingly, the present invention relates to a novel process for making N-aryl pyridones.

The present invention also relates to novel N-aryl pyridones.

The present invention also relates to a novel process for making pyridinolates.

The present invention also relates to novel pyridinolates.

These and other embodiments, which will become apparent during the following detailed description of processes relating to N-aryl pyridones of formula V.

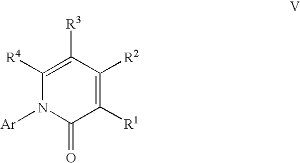

### DETAILED DESCRIPTION OF THE INVENTION

In a first embodiment, the present invention provides a novel process for preparing a pyridinolate of formula III: 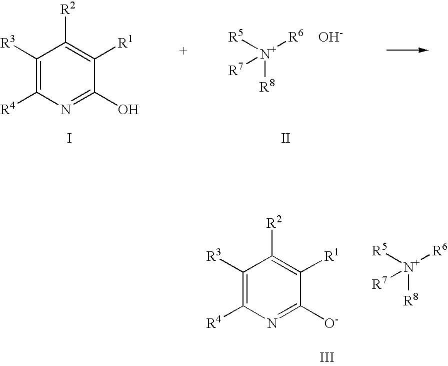
comprising:
(a) contacting a compound of formula I with a compound of formula II under water removing conditions; wherein:
R¹ is selected from H, C₁₋₆ alkyl, OC₁₋₆ alkyl, CF₃, N(C₁₋₆ alkyl)₂, Cl, F, Br, I, phenyl, benzyl, —CN, and NO₂;
R² is selected from H, C₁₋₆ alkyl, OC₁ alkyl, CF₃, N(C₁₋₆ alkyl)₂, Cl, F, Br, I, phenyl, benzyl, —CN, and NO₂;
R³ is selected from H, C₁₋₆ alkyl, OC₁₋₆ alkyl, CF₃, N(C₁₋₆ alkyl)₂, Cl, F, Br, I, phenyl, benzyl, —CN, and NO₂;
R⁴ is selected from H, C₁₋₆ alkyl, OC₁₋₆ alkyl, CF₃, N(C₁₋₆ alkyl)₂, Cl, F, Br, I, phenyl, benzyl, —CN, and NO₂;
R⁵ is selected from C₁₋₂₀ alkyl, phenyl, and benzyl;
R⁶ is selected from C₁₋₈ alkyl, phenyl, and benzyl;
R⁷ is selected from C₁₋₁₈ alkyl, phenyl, and benzyl; and
R⁸ is selected from C₁₋₁₈ alkyl, phenyl, and benzyl.

In a second embodiment, the present invention provides a novel process for preparing a compound of formula III, wherein:
(a) is performed in the presence of a first solvent;
the first solvent is capable of forming an azeotrope;
R¹ is selected from H, C₁₋₄ alkyl, Cl, F, and benzyl;
R² is selected from H, C₁₋₄ alkyl, Cl, F, and benzyl;
R³ is selected from H, C₁₋₄ alkyl, Cl, F, and benzyl;
R⁴ is selected from H, C₁₋₄ alkyl, Cl, F, phenyl, and benzyl;
R⁵ is selected from C₁₋₆ alkyl, phenyl, and benzyl;
R⁶ is selected from C₁₋₆ alkyl, phenyl, and benzyl;
R⁷ is selected from C₁₋₆ alkyl, phenyl, and benzyl; and
R⁸ is selected from C₁₋₆ alkyl, phenyl, and benzyl.

In a third embodiment, the present invention provides a novel process for preparing a compound of formula III, wherein:
the first solvent is selected from toluene and benzene;
R¹ is selected from H and CH₃;
R² is selected from H and CH₃;
R³ is selected from H and CH₃;
R⁴ is selected from H and CH₃;
R⁵ is selected from C₁₋₆ alkyl;
R⁶ is selected from C₁₋₆ alkyl;
R⁷ is selected from C₁₋₆ alkyl; and
R⁸ is selected from C₁₋₆ alkyl.

In a fourth embodiment, the present invention provides a novel process for preparing a compound of formula III, wherein:
the first solvent is toluene;
R¹ is H;
R² is H;
R³ is H;
R⁴ is H;
R⁵ is n-butyl;
R⁶ is n-butyl;
R⁷ is n-butyl; and
R⁸ is n-butyl.

In a fifth embodiment, the present invention provides a novel process for preparing a compound of formula V: 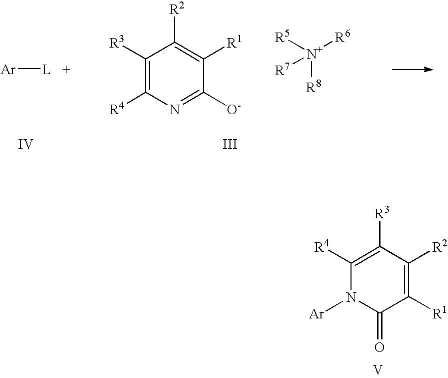
comprising:
(b) contacting a compound of formula IV with a compound of formula III in the presence of a metal salt and a second solvent; wherein:
metal salt is selected from a copper and a palladium salt;
the second solvent is an alcoholic or an aprotic solvent;
R¹ is selected from H, C₁₋₆ alkyl, OC₁₋₆ alkyl, CF₃, N(C₁₋₆ alkyl)₂, Cl, F, Br, I, phenyl, benzyl, —CN, and NO₂;
R² is selected from H, C₁₋₆ alkyl, OC₁₋₆ alkyl, CF₃, N(C₁₋₆ alkyl)₂, Cl, F, Br, I, phenyl, benzyl, —CN, and NO₂;
R³ is selected from H, C₁₋₆ alkyl, OC₁₋₆ alkyl, CF₃, N(C₁₋₆ alkyl)₂, Cl, F, Br, I, phenyl, benzyl, —CN, and NO₂;
R⁴ is selected from H, C₁₋₆ alkyl, OC₁₋₆ alkyl, CF₃, N(C₁₋₆ alkyl)₂, Cl, F, Br, I, phenyl, benzyl, —CN, and NO₂;
R⁵ is selected from C₁₋₂₀ alkyl, phenyl, and benzyl;
R⁶ is selected from C₁₋₁₈ alkyl, phenyl, and benzyl;
R⁷ is selected from C₁₋₁₈ alkyl, phenyl, and benzyl;
R⁸ is selected from C₁₋₁₈ alkyl, phenyl, and benzyl;
L is a leaving group;
Ar is an optionally substituted 5-10 membered aromatic carbocycle or heterocycle consisting of: carbon atoms and 0-4 heteroatoms selected from O, N, and S(O)ₚ; and
p is selected from 0, 1, and 2.

In a sixth embodiment, the present invention provides a novel process for preparing a compound of formula Va: 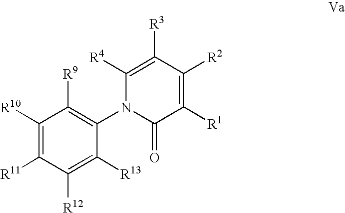
comprising:
(b) contacting a compound of formula IVa with a compound of formula III in the presence of a metal salt and a second solvent; 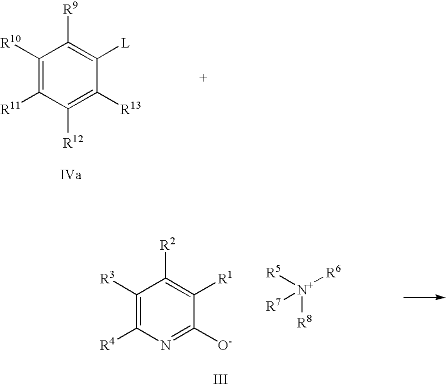
   wherein:
metal salt is a copper (I) salt;
the second solvent is an aprotic solvent;
R¹ is selected from H, C₁₋₄ alkyl, Cl, F, and benzyl;
R² is selected from H, C₁₋₄ alkyl, Cl, F, and benzyl;
R³ is selected from H, C₁₋₄ alkyl, Cl, F, and benzyl;
R⁴ is selected from H, C₁₋₄ alkyl, Cl, F, phenyl, and benzyl;
R⁵ is selected from C₁₋₆ alkyl, phenyl, and benzyl;
R⁶ is selected from C₁₋₆ alkyl, phenyl, and benzyl;
R⁷ is selected from C₁₋₆ alkyl, phenyl, and benzyl;
R⁸ is selected from C₁₋₆ alkyl, phenyl, and benzyl;
L is a leaving group selected from a halogen and a sulfonate;
R⁹ is selected from H, C₁₋₆ alkyl, Cl, and F;
R¹⁰ is selected from H, C₁₋₆ alkyl, phenyl, benzyl, C₁₋₆ alkyl-OH, O—C₁₋₆ alkyl, C(O)—C₁₋₆ alkyl, CO₂—C₁₋₆ alkyl, C(O)NH₂, C(O)NH(C₁₋₄ alkyl), C(O)N(C₁₋₄ alkyl)₂, NHC(O)C₁₋₄ alkyl, N(C₁₋₄ alkyl)C(O)C₁₋₄alkyl, S(O)ₚ—C₁₋₆ alkyl, S(O)ₚNH₂, S(O)ₚNH(C₁₋₄ alkyl), S(O)ₚN(C₁₋₄ alkyl)₂, NH₂, NH(C₁₋₄ alkyl), N(C₁₋₄ alkyl)₂, C₁₋₄ alkylene-NH₂, C₁₋₄ alkylene-NH(C₁₋₄ alkyl), C₁₋₄ alkylene-N(C₁₋₄ alkyl)₂, and NO₂;
R¹¹ is selected from H, C₁₋₆ alkyl, phenyl, benzyl, C₁₋₆ alkyl-OH, O—C₁₋₆ alkyl, C(O)—C₁₋₆ alkyl, CO₂—C₁₋₆ alkyl, C(O)NH₂, C(O)NH(C₁₋₄ alkyl), C(O)N(C₁₋₄ alkyl)₂, NHC(O)C₁₋₄ alkyl, N(C₁₋₄ alkyl)C(O)C₁₋₄ alkyl, S(O)ₚ—C₁₋₆ alkyl, S(O)ₚNH₂, S(O)ₚNH(C₁₋₄ alkyl), S(O)ₚN(C₁₋₄ alkyl)₂, NH₂, NH(C₁₋₄ alkyl), N(C₁₋₄ alkyl)₂, C₁₋₄ alkylene-NH₂, C₁₋₄ alkylene-NH(C₁₋₄ alkyl), C₁₋₄ alkylene-N(C₁₋₄ alkyl)₂, and NO₂;
   R¹² is selected from H, C₁₋₆ alkyl, phenyl, benzyl, C₁₋₆ alkyl-OH, O—C₁₋₆ alkyl, C(O)—C₁₋₆ alkyl, CO₂—C₁₋₆ alkyl, C(O)NH₂, C(O)NH(C₁₋₄ alkyl), C(O)N(C₁₋₄ alkyl)₂, NHC(O)C₁₋₄ alkyl, N(C₁₋₄ alkyl)C(O)C₁₋₄ alkyl, S(O)ₚ—C₁₋₆ alkyl, S(O)ₚNH₂, S(O)ₚNH(C₁₋₄ alkyl), S(O)ₚN(C₁₋₄ alkyl)₂, NH₂, NH(C₁₋₄ alkyl), N(C₁₋₄ alkyl)₂, C₁₋₄ alkylene-NH₂, C₁₋₄ alkylene-NH(C₁₋₄ alkyl), C₁₋₄ alkylene-N(C₁₋₄ alkyl)₂, and NO₂;
   R¹³ is selected from H, C₁₋₆ alkyl, Cl, and F; and
   p, at each occurrence, is selected from 0, 1, and 2.

In a seventh embodiment, the present invention provides a novel process for preparing a compound of formula Va:
metal salt is selected from CuI and CuOTf;
the second solvent is DMF;
R¹ is selected from H and CH₃;
R² is selected from H and CH₃;
R³ is selected from H and CH₃;
R⁴ is selected from H and CH₃;
R⁵ is selected from C₁₋₆ alkyl;
R⁶ is selected from C₁₋₆ alkyl;
R⁷ is selected from C₁₋₆ alkyl;
R⁸ is selected from C₁₋₆ alkyl;
L is a leaving group selected from Cl, Br, I, OSO₂Me, OSO₂CF₃, OSO₂Ph, and OSO₂Ph-p-Me;
R⁹ is selected from H, C₁₋₄ alkyl, Cl, and F;
R¹⁰ is selected from H, C₁₋₄ alkyl, phenyl, benzyl, O—C₁₋₄ alkyl, C(O)—C₁₋₄ alkyl, CO₂—C₁₋₄ alkyl, and NO₂;
R¹¹ is selected from H, C₁₋₄ alkyl, phenyl, benzyl, O—C₁₋₄ alkyl, C(O)—C₁₋₄ alkyl, CO₂—C₁₋₄ alkyl, NH₂, and NO₂;
R¹² is selected from H, C₁₋₄ alkyl, phenyl, benzyl, O—C₁₋₄ alkyl, C(O)—C₁₋₄ alkyl, CO₂—C₁₋₄ alkyl, and NO₂; and
R¹³ is selected from H, C₁₋₄ alkyl, Cl, and F.

In an eighth embodiment, the present invention provides a novel process for preparing a compound of formula V:
metal salt is CuI;
the second solvent is DMF;
R¹ is H;
R² is H;
R³ is H;
R⁴ is H;
R⁵ is n-butyl;
R⁶ is n-butyl;
R⁷ is n-butyl;
R⁸ is n-butyl;
L is I;
R⁹ is H;
R¹⁰ is selected from H, CH₃, CH₂CH₃, CH(CH₃)₂, and NO₂;
R¹¹ is selected from H, C₁₋₄ alkyl, OCH₃, CO₂CH₂CH₃, NH₂, and NO₂;
R¹² is selected from H, C₁₋₄ alkyl, and NO₂; and
R¹³ is H.

In a ninth embodiment, the present invention provides a novel pyridinolate of formula III: 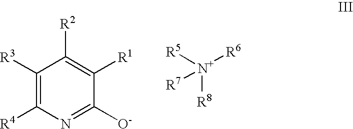
wherein:
R¹ is selected from H, C₁₋₆ alkyl, OC₁₋₆ alkyl, CF₃, N(C₁₋₆ alkyl)₂, Cl, F, Br, I, phenyl, benzyl, —CN, and NO₂;
R² is selected from H, C₁₋₆ alkyl, OC₁₋₆ alkyl, CF₃, N(C₁₋₆ alkyl)₂, Cl, F, Br, I, phenyl, benzyl, —CN, and NO₂;
R³ is selected from H, C₁₋₆ alkyl, OC₁₋₆ alkyl, CF₃, N(C₁₋₆ alkyl)₂, Cl, F, Br, I, phenyl, benzyl, —CN, and NO₂;
R⁴ is selected from H, C₁₋₆ alkyl, OC₁₋₆ alkyl, CF₃, N(C₁₋₆ alkyl)₂, Cl, F, Br, I, phenyl, benzyl, —CN, and NO₂;
R⁵ is selected from C₁₋₂₀ alkyl, phenyl, and benzyl;
R⁶ is selected from C₁₋₁₈ alkyl, phenyl, and benzyl;
R⁷ is selected from C₁₋₁₈ alkyl, phenyl, and benzyl; and
R⁸ is selected from C₁₋₁₈ alkyl, phenyl, and benzyl.

In a tenth embodiment, the present invention provides a novel compound of formula Va: 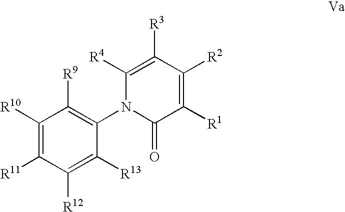
wherein:
R¹ is selected from H, C₁₋₄ alkyl, Cl, F, and benzyl;
R² is selected from H, C₁₋₄ alkyl, Cl, F, and benzyl;
R³ is selected from H, C₁₋₄ alkyl, Cl, F, and benzyl;
R⁴ is selected from H, C₁₋₄ alkyl, Cl, F, phenyl, and benzyl;
R⁹ is selected from H, C₁₋₆ alkyl, Cl, and F;
R¹⁰ is selected from H, C₁₋₆ alkyl, phenyl, benzyl, C₁₋₆ alkyl-OH, O—C₁₋₆ alkyl, C(O)—C₁₋₆ alkyl, CO₂—C₁₋₁₆ alkyl, C(O)NH₂, C(O)NH(C₁₋₄ alkyl), C(O)N(C₁₋₄ alkyl)₂, NHC(O)C₁₋₄ alkyl, N(C₁₋₄ alkyl)C(O)C₁₋₄ alkyl, S(O)ₚ—C₁₋₆ alkyl, S(O)ₚNH₂, S(O)ₚNH(C₁₋₄ alkyl), S(O)ₚN(C₁₋₄ alkyl)₂, NH₂, NH(C₁₋₄ alkyl), N(C₁₋₄ alkyl)₂, C₁₋₄ alkylene-NH₂, C₁₋₄ alkylene-NH(C₁₋₄ alkyl), C₁₋₄ alkylene-N(C₁₋₄ alkyl)₂, and NO₂;
R¹¹ is selected from H, C₁₋₆ alkyl, phenyl, benzyl, C₁₋₆ alkyl-OH, O—C₁₋₆ alkyl, C(O)—C₁₋₆ alkyl, CO₂—C₁₋₆ alkyl, C(O)NH₂, C(O)NH(C₁₋₄ alkyl), C(O)N(C₁₋₄ alkyl)₂, NHC(O)C₁₋₄ alkyl, N(C₁₋₄ alkyl)C(O)C₁₋₄ alkyl, S(O)ₚ—C₁₋₆ alkyl, S(O)ₚNH₂, S(O)ₚNH(C₁₋₄ alkyl), S(O)ₚN(C₁₋₄ alkyl)₂, NH₂, NH(C₁₋₄ alkyl), N(C₁₋₄ alkyl)₂, C₁₋₄ alkylene-NH₂, C₁₋₄ alkylene-NH(C₁₋₄ alkyl), C₁₋₄ alkylene-N(C₁₋₄ alkyl)₂, and NO₂;
R¹² is selected from H, C₁₋₆ alkyl, phenyl, benzyl, C₁₋₆ alkyl-OH, O—C₁₋₆ alkyl, C(O)—C₁₋₆ alkyl, CO₂—C₁₋₁₆ alkyl, C(O)NH₂, C(O)NH(C₁₋₄ alkyl), C(O)N(C₁₋₄ alkyl)₂, NHC(O)C₁₋₄ alkyl, N(C₁₋₄ alkyl)C(O)C₁₋₄ alkyl, S(O)ₚ—C₁₋₆ alkyl, S(O)ₚNH₂, S(O)ₚNH(C₁₋₄ alkyl), S(O)ₚN(C₁₋₄ alkyl)₂, NH₂, NH(C₁₋₄ alkyl), N(C₁₋₄ alkyl)₂, C₁₋₄ alkylene-NH₂, C₁₋₄ alkylene-NH(C₁₋₄ alkyl), C₁₋₄ alkylene-N(C₁₋₄ alkyl)₂, and NO₂;
R¹³ is selected from H, C₁₋₆ alkyl, Cl, and F; and
p, at each occurrence, is selected from 0, 1, and 2.

The present invention may be embodied in other specific forms without departing from the spirit or essential attributes thereof. Thus, the above embodiments should not be considered limiting. Any and all embodiments of the present invention may be taken in conjunction with any other embodiment or embodiments to describe additional embodiments. Each individual element of the embodiments is its own independent embodiment. Furthermore, any element of an embodiment is meant to be combined with any and all other elements from any embodiment to describe an additional embodiment. In addition, the present invention encompasses combinations of different embodiment, parts of embodiments, definitions, descriptions, and examples of the invention noted herein.

Definitions

All examples provided in the definitions as well as in other portions of this application are not intended to be limiting, unless stated.

The present invention can be practiced on multigram scale, kilogram scale, multikilogram scale, or industrial scale. Multigram scale, as used herein, is can be in the scale wherein at least one starting material is present in 10 grams or more, at least 50 grams or more, or at least 100 grams or more. Multikilogram scale means the scale wherein more than one kilo of at least one starting material is used. Industrial scale means a scale which is other than a laboratory sale and which is sufficient to supply product sufficient for either clinical tests or distribution to consumers.

Equivalents mean molar equivalents unless otherwise specified.

The compounds herein described may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, and racemic forms and all geometric isomeric forms of a structure are intended, unless the specific stereochemistry or isomeric form is specifically indicated. All processes used to prepare compounds of the present invention and intermediates made therein are considered to be part of the present invention. Tautomers of compounds shown or described herein are considered to be part of the present invention.

Examples of the molecular weight of compounds of the present invention include (a) less than about 500, 550, 600, 650, 700, 750, or 800 grams per mole, (b) 800 grams per mole, (c) less than about 750 grams per mole, and (d) less than about 700 grams per mole.

“Substituted” means that any one or more hydrogens on the designated atom is replaced with a selection from the indicated group, provided that the designated atom's normal valency is not exceeded, and that the substitution results in a stable compound. When a substituent is keto (i.e., ═O), then 2 hydrogens on the atom are replaced. Keto substituents are not present on aromatic moieties.

The present invention includes all isotopes of atoms occurring in the present compounds. Isotopes include those atoms having the same atomic number but different mass numbers. By way of general example and without limitation, isotopes of hydrogen include tritium and deuterium. Isotopes of carbon include C-13 and C-14.

The present invention is also includes all stable oxides of thiol and amino groups, even when not specifically written. When an amino group is listed as a substituent, the N-oxide derivative of the amino group is also included as a substituent. When a thiol group is present, the S-oxide and S,S-dioxide derivatives are also included.

When any variable (e.g., R⁶) occurs more than one time in any constituent or formula for a compound, its definition at each occurrence is independent of its definition at every other occurrence. Thus, for example, if a group is substituted with 0-2 R⁶, then the group may optionally be substituted with up to two R⁶ groups and R⁶ at each occurrence is selected independently from the definition of R⁶. Also, combinations of substituents and/or variables are permissible only if such combinations result in stable compounds.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

Suitable aprotic solvents include ether solvents, dimethylformamide (DMF), dimethylacetamide (DMAC), benzene, toluene, 1,3-dimethyl-3,4,5,6-tetrahydro-2(1H)-pyrimidinone (DMPU), 1,3-dimethyl-2-imidazolidinone (DMI), N-methylpyrrolidinone (NMP), formamide, N-methylacetamide, N-methylformamide, acetonitrile, dimethyl sulfoxide, propionitrile, ethyl formate, methyl acetate, hexachloroacetone, acetone, ethyl methyl ketone, ethyl acetate, sulfolane, N,N-dimethylpropionamide, tetramethylurea, nitromethane, nitrobenzene, or hexamethylphosphoramide.

Alcoholic solvents can be C₁₋₆ alkyl groups with 1 hydroxy group. The alkyl groups can be linear or branched. Alcoholic solvents covers primary (e.g., methanol), secondary (e.g., isopropanol alcohol), and tertiary (e.g., 2-methyl-2-propanol) alcohols. Suitable alcoholic solvents include methanol, ethanol, 1-propanol, 2-propanol, 1-butanol, 2-butanol, 2-methyl-1-propanol, 2-methyl-2-propanol, 1-pentanol, 2-pentanol, 3-pentanol, 2,2-dimethyl-1-propanol, 3-methylbutanol, 2-methyl-2-butanol, 1-hexanol, and 2-ethyl-1-butanol.

Suitable ether solvents include dimethoxymethane, tetrahydrofuran, 1,3-dioxane, 1,4-dioxane, furan, diethyl ether, 1,2-dimethoxyethane, diethoxymethane, dimethoxymethane, ethylene glycol dimethyl ether, ethylene glycol diethyl ether, diethylene glycol dimethyl ether, diethylene glycol diethyl ether, triethylene glycol dimethyl ether, or t-butyl methyl ether.

“Alkyl” and “alkylene” includes both branched and straight-chain saturated aliphatic hydrocarbon groups having the specified number of carbon atoms. C₁₋₁₀ alkyl, includes C₁, C₂, C₃, C₄, C₅, C₆, C₇, C₈, C₉, and C₁₀ alkyl groups. Examples of alkyl include methyl, ethyl, n-propyl, i-propyl, n-butyl, s-butyl, t-butyl, n-pentyl, and s-pentyl. Examples of alkylene include methylene, ethylene, n-propylene, i-propylene, n-butylene, s-butylene, t-butylene, n-pentylene, and s-pentylene. “Haloalkyl” includes both branched and straight-chain saturated aliphatic hydrocarbon groups having the specified number of carbon atoms, substituted with 1 or more halogen (for example —CᵥF_{w} where v=1 to 3 and w=1 to (2v+1)). Examples of haloalkyl include trifluoromethyl, trichloromethyl, pentafluoroethyl, and pentachloroethyl. “Alkoxy” represents an alkyl group as defined above with the indicated number of carbon atoms attached through an oxygen bridge. C₁₋₁₀ alkoxy, includes C₁, C₂, C₃, C₄, C₅, C₆, C₇, C₈, C₉, and C₁₀ alkoxy groups. Examples of alkoxy include methoxy, ethoxy, n-propoxy, i-propoxy, n-butoxy, s-butoxy, t-butoxy, n-pentoxy, and s-pentoxy. “Cycloalkyl” includes saturated ring groups, such as cyclopropyl, cyclobutyl, or cyclopentyl. C₃₋₇ cycloalkyl includes C₃, C₄, C₅, C₆, and C₇ cycloalkyl groups. Alkenyl” includes hydrocarbon chains of either straight or branched configuration and one or more unsaturated carbon-carbon bonds that may occur in any stable point along the chain, such as ethenyl and propenyl. C₂₋₁₀ alkenyl includes C₂, C₃, C₄, C₅, C₆, C₇, C₈, C₉, and C₁₀ alkenyl groups. “Alkynyl” includes hydrocarbon chains of either straight or branched configuration and one or more triple carbon-carbon bonds that may occur in any stable point along the chain, such as ethynyl and propynyl. C₂₋₁₀ Alkynyl includes C₂, C₃, C₄, C₅, C₆, C₇, C₈, C₉, and C₁₀ alkynyl groups.

“Halo” or “halogen” refers to fluoro, chloro, bromo, and iodo; and “counterion” is used to represent a small, negatively charged species such as chloride, bromide, hydroxide, acetate, and sulfate.

“Carbocycle” means any stable 3, 4, 5, 6, or 7-membered monocyclic or bicyclic or 7, 8, 9, 10, 11, 12, or 13-membered bicyclic or tricyclic, any of which may be saturated, partially unsaturated, or unsaturated (aromatic). When a carbocycle is referred to as an “aromatic” or “aromatic carbocycle,” this means that a fully unsaturated, i.e., aromatic, ring is present in the carbocycle. An aromatic carboocycle only requires one ring to be aromatic, if more than one ring is present (e.g., tetrahydronaphthalene). Examples of such carbocycles include cyclopropyl, cyclobutyl, cyclopentyl, cyclohexyl, cycloheptyl, adamantyl, cyclooctyl, [3.3.0]bicyclooctane, [4.3.0]bicyclononane, [4.4.0]bicyclodecane, [2.2.2]bicyclooctane, fluorenyl, phenyl, naphthyl, indanyl, adamantyl, and tetrahydronaphthyl.

“Heterocycle” or “heterocyclic group” means a stable 3, 4, 5, 6, or 7-membered monocyclic or 7, 8, 9, 10, 11, or 12-membered bicyclic or tricyclic heterocyclic ring which is saturated, partially unsaturated, or unsaturated (aromatic), and which consists of carbon atoms and 1, 2, 3, 4, or 5 ring heteroatoms independently selected from the group consisting of N, O and S. Heterocycle includes any bicyclic group in which one heterocyclic ring is fused to a second ring, which may be carbocyclic (e.g. benzo fusion) or heterocyclic. When a heterocycle is referred to as an “aromatic heterocycle” or “heteroaryl,” this means that a fully unsaturated, i.e., aromatic, ring is present in the heterocycle. An aromatic heterocycle only requires one ring to be aromatic, if more than one ring is present. The aromatic portion of the aromatic heterocycle can be a carbocycle or heterocycle. The nitrogen and sulfur heteroatoms in the heterocycle may optionally be oxidized (i.e., N→O and S(O)p). The nitrogen atom may be unsubstituted (i.e., N or NH) or substituted (i.e., NR wherein R is a substituent) and may optionally be quaternized. The heterocyclic ring may be attached to its pendant group at any heteroatom or carbon atom that results in a stable structure. The heterocyclic rings described herein may be substituted on a carbon or on a nitrogen atom, if the resulting compound is stable. If the total number of S and O atoms in the heterocycle exceeds 1, then these heteroatoms can be non-adjacent. As an example, the total number of S and O atoms in the heterocycle can be 0 or 1. Bridged and spiro rings are also included in the definition of heterocycle. A bridged ring occurs when one or more atoms (i.e., C, O, N, or S) link two non-adjacent carbon or nitrogen atoms. Examples of bridges include one carbon atom, two carbon atoms, one nitrogen atom, two nitrogen atoms, and a carbon-nitrogen group. It is noted that a bridge always converts a monocyclic ring into a tricyclic ring. When a ring is bridged, the substituents recited for the ring may also be present on the bridge. Spiro rings are formed when to or more atoms (i.e., C, O, N, or S) of a chain are attached to the same carbon atom of a heterocycle (or carbocycle if fused to a heterocycle). When a spiro ring is present, the substituents recited for the ring may also be present on the spiro.

Examples of heterocycles include, but are not limited to, acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, iindazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl. Also included are fused ring and spiro compounds containing, for example, the above heterocycles.

Optionally substituted covers from 0-5 substituents selected from H, C₁₋₆ alkyl, phenyl, benzyl, C₁₋₆ alkyl-OH, O—C₁₋₆ alkyl, C(O)—C₁₋₆ alkyl, CO₂—C₁₋₆ alkyl, C(O)NH₂, C(O)NH(C₁₋₄ alkyl), C(O)N(C₁₋₄ alkyl)₂, NHC(O)C₁₋₄ alkyl, N(C₁₋₄ alkyl)C(O)C₁₋₄ alkyl, S(O)ₚ—C₁₋₆ alkyl, S(O)ₚNH₂, S(O)ₚNH(C₁₋₄ alkyl), S(O)ₚN(C₁₋₄ alkyl)₂, NH₂, NH(C₁₋₄ alkyl), N(C₁₋₄ alkyl)₂, C₁₋₄ alkylene-NH₂, C₁₋₄ alkylene-NH(C₁₋₄ alkyl), C₁₋₄ alkylene-N(C₁₋₄ alkyl)₂, and NO₂;

“Stable compound” and “stable structure” indicate a compound that is sufficiently robust to survive isolation to a useful degree of purity from a reaction mixture, and formulation into an efficacious therapeutic agent.

“Substituted” indicates that one or more hydrogens on the atom indicated in the expression using “substituted” is replaced with a selection from the indicated group(s), provided that the indicated atom's normal valency is not exceeded, and that the substitution results in a stable compound. When a substituent is keto (i.e., ═O) group, then 2 hydrogens on the atom are replaced.

Synthesis

By way of example and without limitation, the present invention may be further understood by the following schemes and descriptions.

### Preparation of Pyridin-2-olates

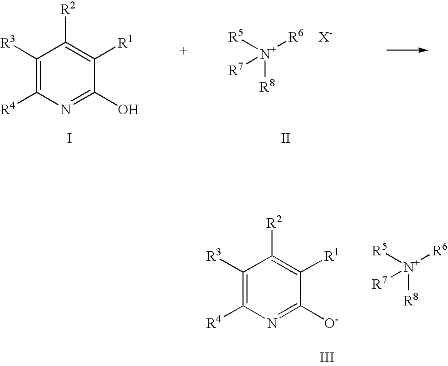 Reaction (a)

The 2-pyridinium oxide salt, III, can be made from its corresponding hydroxy-pyridine (I) and ammonium salt (II) (e.g., an ammonium hydroxide salt). The hydroxy-pyridine and hydroxy-ammonium salt can be contacted in solvent capable of forming an azeotrope (e.g., toluene and benzene) under water removing conditions (e.g., Dean-Stark apparatus or distallation). This reaction can be run from room temperature up to the reflux point of the solvent used. The 2-pyridinium oxide salt, once formed, can be used in situ or can be isolated prior to contacting with formula IV.

Suitable examples of ammonium hydroxides and the corresponding pyridin-2-olate include, but are not limited to: benzyltrimethylammonium hydroxide (to form benzyltrimethylammonium pyridin-2-olate), diethyldimethylammonium hydroxide (to form diethyldimethylammonium pyridin-2-olate), dimethyldodecylethylammonium hydroxide (to form dimethyldodecylethylammonium pyridin-2-olate), hexadecyltrimethylammonium hydroxide (to form hexadecyltrimethylammonium pyridin-2-olate), methyltripropylammonium hydroxide (to form methyltripropylammonium pyridin-2-olate), tetrabutylammonium hydroxide (to form tetrabutylammonium pyridin-2-olate), tetraethylammonium hydroxide (to form tetraethylammonium pyridin-2-olate), tetrahexylammonium hydroxide (to form tetrahexylammonium pyridin-2-olate), tetrakis (decyl)ammonium hydroxide (to form tetrakis (decyl)ammonium pyridin-2-olate), tetramethylammonium hydroxide (to form tetramethylammonium pyridin-2-olate), tetraoctadecylammonium hydroxide (to form tetraoctadecylammonium pyridin-2-olate), tetraoctylammonium hydroxide (to form tetraoctylammonium pyridin-2-olate), tetrapentylammonium hydroxide (to form tetrapentylammonium pyridin-2-olate), tetrapropylammonium hydroxide (to form tetrapropylammonium pyridin-2-olate), trimethylphenylammonium hydroxide (to form trimethylphenylammonium pyridin-2-olate), tributylmethylammonium hydroxide (to form tributylmethylammonium pyridin-2-olate), triethylmethylammonium hydroxide (to form triethylmethylammonium pyridin-2-olate), trihexyltetradecylammonium hydroxide (to form trihexyltetradecylammonium pyridin-2-olate), and trimethylphenylammonium hydroxide (to form trimethylphenylammonium pyridin-2-olate).
Pyridone Formation 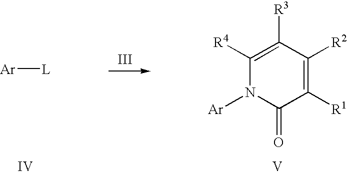
Reaction (b)

Formula V can be formed by reacting formula IV with 2-pyridinium oxide salt III. The aromatic ring of formula IV may be substituted with from 1-5 substituents. The only limitation is that the substituent(s) can not be a group that will interfere with reaction (b). Reaction (b) can be conducted in the presence of a metal salt catalyst. Examples of metal salt catalysts include (a) a copper salt (e.g., CuI, CuCl, CuBr, and CuOTf) or a palladium salt (e.g., PdCl₂ and Pd(OAc)₂), (b) a copper (I) salt, and (c) CuI or CuOTf. This reaction can be run in a number of solvents, including alcohols and aprotic solvents. Examples of solvents for the reaction include (a) alcohols and aprotic solvents, (b) aprotic solvents, and (c) DMF. Examples of reaction temperatures include (a) from room temperature up to the reflux point of the solvent used, (b) from about room temperature, 30, 40, 50, 60, 70, 80, 90, 100, 110, 120, 130, 140, 150, to 160° C., and (c) from room temperature to about 160° C. It may be useful to run this reaction under an inert atmosphere (e.g., nitrogen or argon).

Examples of compounds that can be prepared using the above-described pyrdinolates include those shown below. The enantiomer not shown can also be prepared.

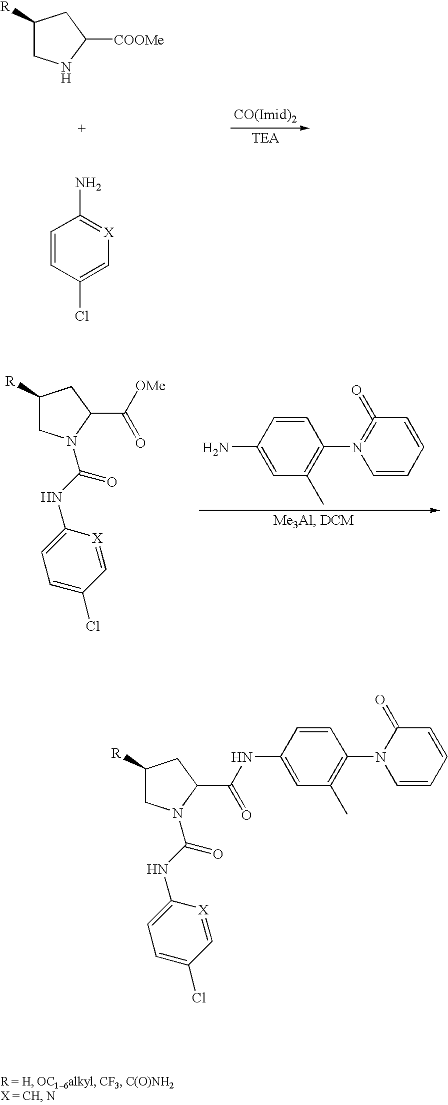

Additional examples of compounds that can be prepared using the above-described pyrdinolates include those shown below. Both enantiomers for the compounds shown below can be prepared by this methodology.

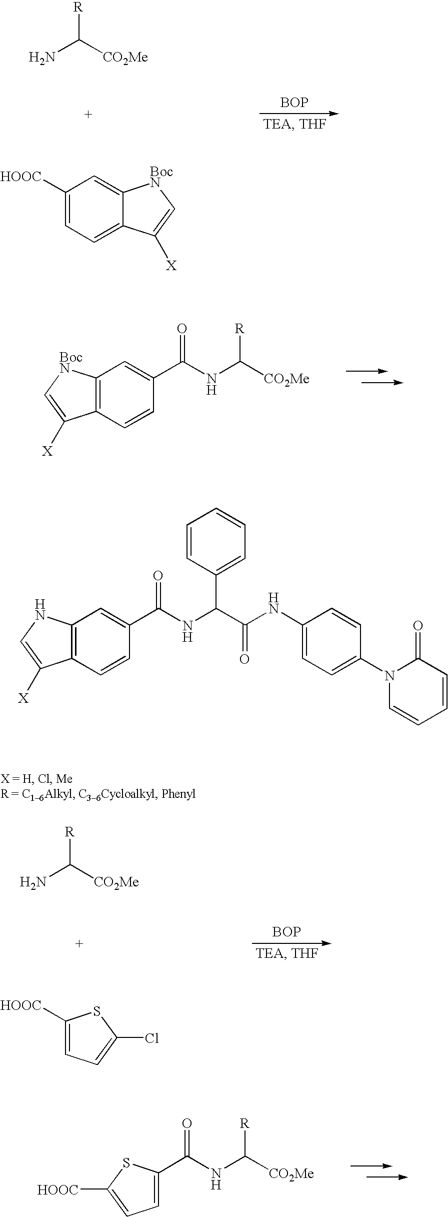

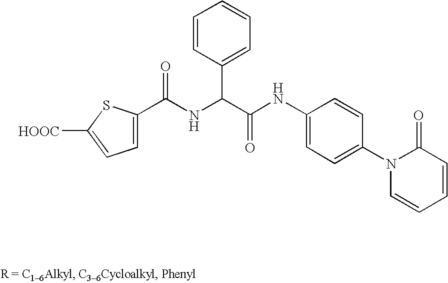

More examples of compounds that can be prepared using the above-described pyrdinolates are shown in the following two schemes.

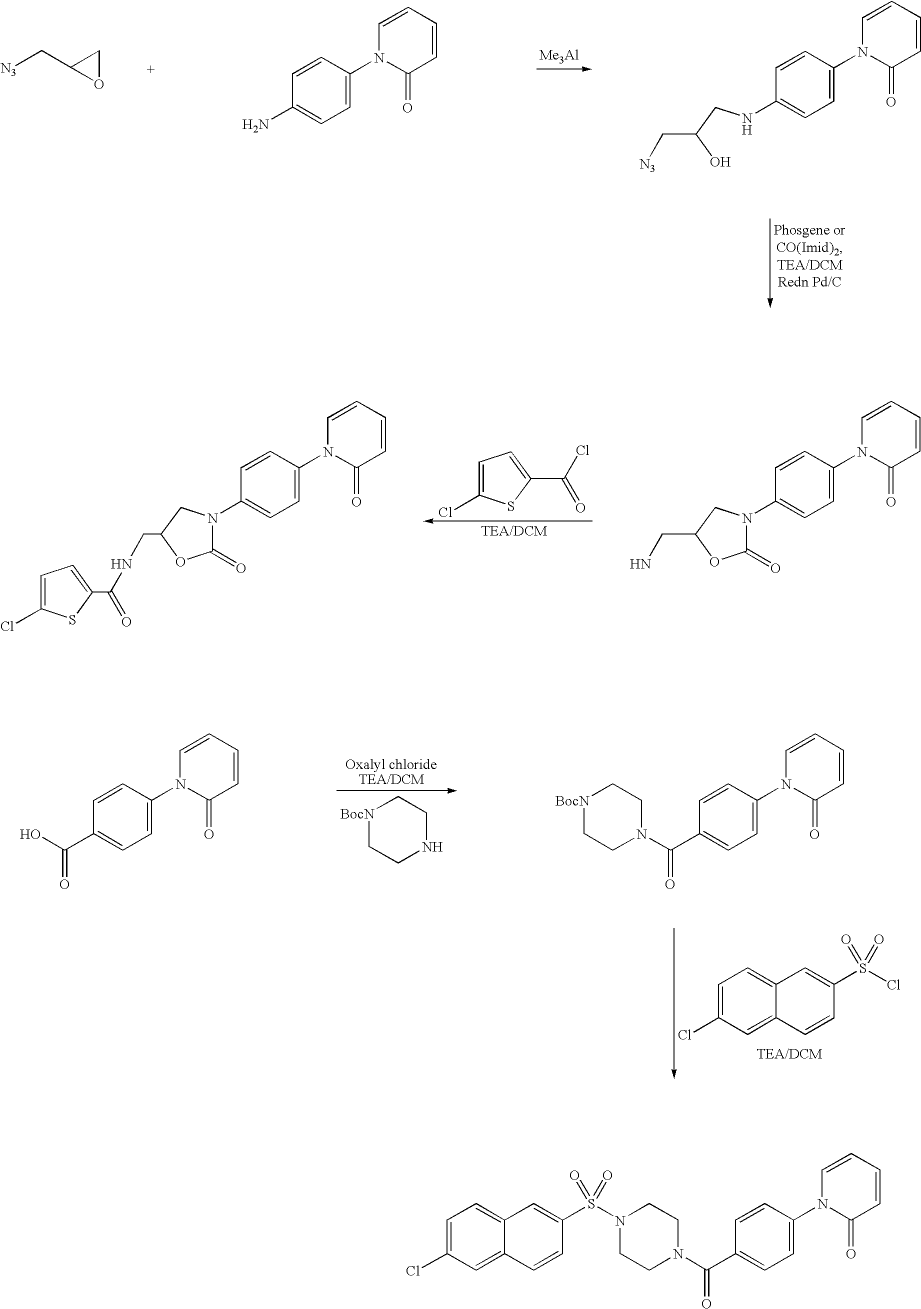

Other features of the invention will become apparent in the course of the following descriptions of examplary embodiments which are given for illustration of the invention and are not intended to be limiting thereof.

### EXAMPLES

### Example 1

### Tetrabutylammonium pyridin-2-olate

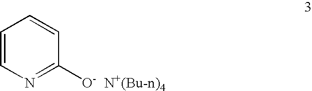

Method A: A 1L round bottom flask was charged with 2-pyridone (47.5 g, 0.5 mol, 1 eq), tetrabutyl ammonium hydroxide (40% of aqueous solution, 324.3 g, 0.5 mol, 1 eq), and toluene (300 mL). The water was removed via a Dean-Stark apparatus. After all water was removed, the solution was cooled to rt and then to 0° C. and remained at 0° C. for 30 minutes. The slurry was filtrated under N₂ and the solid was dried under vacuum over P₂O₅ at 50° C. for 12 hours to afford the desired product as a solid (68 g, 38%).

Method B: To a 1L round bottom flask was charged with 2-pyridone (47.5 g, 0.5 mol, 1 eq) and tetrabutyl ammonium hydroxide (40% of aqueous solution, 324.3 g, 0.5 mol, 1 eq) and toluene (300 mL). The solvent was distilled under reduced pressure at 55° C. The residual water was removed azeotropically with toluene (3×300 mL) to afford an amber oil which changed into white solid once cooled to rt. The solid was then dried under vacuum over P₂O₅ at 50° C. for 12 hours to afford the desired product as a solid (173 g, 100%).

¹H NMR (CDCl₃): δ 7.47 (m, 3H); 7.37-7.26 (m, 6H); 7.20 (dd, J=7.3, 1.7 Hz, 1H); 6.94 (ddd, J=9.2, 3.2, 2.2 Hz, 2H); 6.88 (br s, 1H), 5.73 (br s, 1H), 4.18 (t, J=6.6 Hz, 2H); 3.82 (s, 3H), 3.69 (s, 3H—), 3.41 (t, J=6.6 Hz, 2H); 2.34 (s, 3H); 1.45 (br s, 1H); ¹H NMR (d₆-DMSO): δ 7.75 (s, 1H), 7.54-7.28 (m, 10H), 7.21 (d, J=7.0 Hz, 2H); 6.99 (d, J=7.3 Hz, 2H); 4.11 (br t, J=5.8 Hz, 2H); 3.81 (s, 3H); 3.55 (s, 2H); 3.34 (br s, 1H), 3.23 (br t, J=5.8 Hz, 2H); 2.22 (s, 3H); ¹³C NMR (CDCl₃): δ 167.53, 139.98, 118.68, 106.22, 58.99, 29.57, 20.01, 14.01.

### Example 2

### Ethyl 4-(2-oxopyridin-1(2H)-yl)benzoate

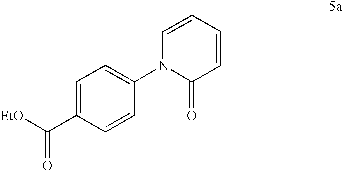

A 50 mL round bottom flask was charged with ethyl 4-iodobenzoate (2.76 g, 10 mmol) and tetrabutylammonium pyridin-2-olate (5.19 g, 15 mmol). A trace of water was removed azeotropically with toluene (2×20 mL). CuI (950 mg, 5 mmol) and DMF (10 mL) were added. The reaction mixture was heated to 120° C. for 12 hours under N₂. The mixture was then cooled to rt. A solid precipitated during the cooling process. The slurry was transferred slowly to aq. NH₄OH (50 mL, 3N). The solid was collected by filtration. The solid was re-dissolved in CH₂Cl₂ (50 mL) and washed with NH₄OH (2×25 mL, 3N) and H₂O (3×30 mL). The organic solution was concentrated in vacuo to provide the desired compound (2.2 g, 90.5%) as a solid. ¹H NMR (CDCl₃): δ 7.94 (d, J=8.4 Hz 2H); 7.25 (d, J=8.4 Hz, 2H); 7.18 (d, J=7.8 Hz, 1H); 7.09 (d, J=8.6 Hz, 1H); 6.43 (d, J=9.3 Hz, 1H), 6.43 (d, J=6.7 Hz, 1H), 4.18 (q, J=7.1 Hz, 2H); 1.19 (t, J=7.1 Hz, 3H).

### Example 3

### 1-(4-Methoxyphenyl)pyridin-2(1H)-one

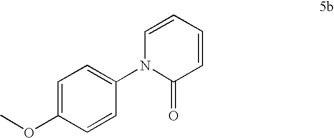

Method A: A 25 mL round bottom flask was charged with 1-iodo-4-methoxybenzene (234 mg, 1 mmol) and tetrabutylammonium pyridin-2-olate (692 mg, 2 mmol). A trace of water was removed azeotropically with toluene (2×10 mL). CuI (95 mg, 0.5 mmol) and DMF (5 mL) were added. The reaction mixture was heated to 110° C. for 12 hours under N₂. The mixture was then cooled to rt. A solid precipitated during the cooling process. The slurry was transferred slowly to aq. NH₄OH (5 mL, 3N). The solid was collected by filtration. The solid was re-dissolved in CH₂Cl₂ (15 mL) and washed with NH₄OH (2×5 mL, 3N) and H₂O (3×5 mL). The organic solution was concentrated in vacuo to provide the desired compound (183 mg, 91%) as a solid. ¹H NMR (CDCl₃): δ 7.29 (m, 1H); 7.22 (m, 3H), 6.91(d, J=11.9 Hz, 2H); 6.55 (d, J=9.1 Hz, 1H); 6.13 (t, J=7.1 Hz, 2H); 3.75 (s, 3H). ¹³C NMR (CDCl₃): δ 163.44, 160.29, 140.7, 128.51, 122.65, 115.44, 106.7, 56.71.

Method B: A 50 mL round bottom flask was charged with 1-iodo-4-methoxybenzene (234 mg, 1 mmol), 2-pyridone (190 mg, 2 mmol), tetrabutyl ammonium chloride (84 mg, 0.3 mmol), NaH (48 mg, 2 mmol), CuI (95 mg, 0.5 mmol), and DMF (5 mL) at rt under N₂. The reaction mixture was heated to 110° C. for 12 hours under N₂. The mixture was then cooled to rt. A solid precipitated during the cooling process. The slurry was transferred slowly to aq. NH₄OH (5 mL, 3N). The solid was collected by filtration. The solid was re-dissolved in CH₂Cl₂ (15 mL) and washed with NH₄OH (2×5 mL, 3N), and H₂O (3×5 mL). The organic solution was concentrated in vacuo to provide the desired compound (178 mg, 89%) as light yellow solid. ¹H NMR (CDCl₃): δ 7.29 (m, 1H); 7.22 (m, 3H), 6.91(d, J=11.9 Hz, 2H); 6.55 (d, J=9.1 Hz, 1H); 6.13 (t, J=7.1 Hz, 2H); 3.75 (s, 3H). ¹³CNMR(CDCl₃): δ 163.44, 160.29, 140.7, 128.51, 122.65, 115.44, 106.7, 56.71.

### Example 4

### 1-(4-Nitrophenyl)pyridin-2(1H)-one

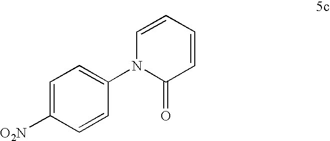

A 25 mL round bottom flask was charged with 1-iodo-4-nitrobenzene (498 mg, 2 mmol) and tetrabutylammonium pyridin-2-olate (1.38 g, 4 mmol). A trace of water was removed azeotropically with toluene (2×10 mL). CuI (190 mg, 1 mmol) and DMF (5 mL) were added. The reaction mixture was heated to 110° C. for 12 hours under N₂. The mixture was then cooled to rt. A solid precipitated during the cooling process. The slurry was transferred slowly to aq. NH₄OH (5 mL, 3N). The solid was collected by filtration. The solid was re-dissolved in CH₂Cl₂ (15 mL) and washed with NH₄OH (2×5 mL, 3N) and H₂O (3×5 mL). The organic solution was concentrated in vacuo to provide the desired compound (393 mg, 91%) as light yellow solid. ¹H NMR (CDCl₃): δ 8.30 (d, J=9.2 Hz, 2H); 7.56 (d, J=9.2 Hz, 2H), 7.37(t, J=7.0 Hz, 1H); 7.26 (d, J=6.7 Hz, 1H); 6.61 (d, J=9.5 Hz, 1H); 6.25 (t, J=6.4 Hz, 1H).

### Example 5

### 1-(4-Isopropylphenyl)pyridin-2(1H)-one

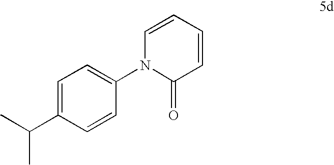

A 50 mL round bottom flask was charged with 1-iodo-4-isopropylbenzene (2.46 g, 10 mmol) and tetrabutylammonium pyridin-2-olate (6.92 g, 20 mmol). A trace of water was removed azeotropically with toluene (2×20 mL). CuI (950 mg, 5 mmol) and DMF (20 mL) were added. The reaction mixture was heated to 110° C. for 12 hours under N₂. The mixture was then cooled to rt. A solid precipitated during the cooling process. The slurry was transferred slowly to aq. NH₄OH (50 mL, 3N). The solid was collected by filtration. The solid was re-dissolved in CH₂Cl₂ (75 mL) and washed with NH₄OH (2×25 mL, 3N) and H₂O (3×50 mL). The organic solution was concentrated in vacuo to provide the desired compound (2.02 g, 95%) as a white solid. ¹H NMR (CDCl₃): δ 7.34 (m, 6H); 6.66 (d, J=9.3 Hz, 1H), 6.22(t, J=7.2 Hz, 1H); 2.97 (m, 1H); 1.28 (d, J=6.9 Hz, 6H). ¹³C NMR (CDCl₃): δ 162.92, 149.55, 140.1, 139.0, 138.5, 127.7, 126.6, 122.2, 106.1, 34.2, 24.3.m/e 214.28, 215.32.

### Example 6

### 1-(4-Amino-3-methyl-5-nitrophenyl)pyridin-2(1H)-one

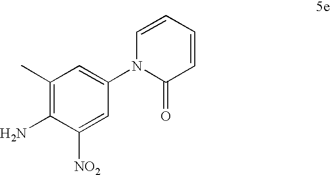

A 25 mL round bottom flask was charged with 4-iodo-2-methyl-6-nitrobenzenamine (278 mg, 1 mmol) and tetrabutylammonium pyridin-2-olate (692 mg, 2 mmol). A trace of water was removed azeotropically with toluene (2×20 mL). CuI (95 mg, 0.5 mmol) and DMF (5 mL) were added. The reaction mixture was heated to 110° C. for 12 hours under N₂. The mixture was then cooled to rt. A solid precipitated during the cooling process. The slurry was transferred slowly to aq. NH₄OH (10 mL, 3N). The solid was collected by filtration. The solid was re-dissolved in CH₂Cl₂ (15 mL) and washed with NH₄OH (2×5 mL, 3N) and H₂O (3×5 mL). The organic solution was concentrated in vacuo to provide the desired compound (218 mg, 89%) as a white solid.

¹H NMR (CDCl₃): δ 7.97 (s, 1H); 7.36 (bs, 2H), 7.26(d, J=6.2 Hz, 1H); 6.59(d, J=9.12 Hz, 1H); 6.33(bs, 2H); 6.20(t, J=6.28 Hz, 1H).

Numerous modifications and variations of the present invention are possible in light of the above teachings. It is therefore to be understood that within the scope of the appended claims, the invention may be practiced otherwise than as specifically described herein.

What is claimed is:

1. A process for preparing a pyridinolate of formula III: 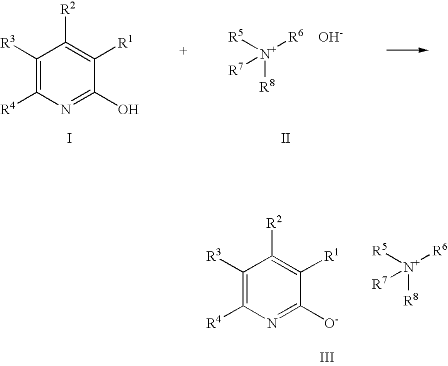
comprising:
(a) contacting a compound of formula I with a compound of formula II under water removing conditions; wherein:
R₁ is selected from H, C₁₋₆ alkyl, OC₁₋₆ alkyl, CF₃, N(C₁₋₆ alkyl)₂, Cl, F, Br, I, phenyl, benzyl, —CN, and NO₂;
R² is selected from H, C₁₋₆ alkyl, OC₁₋₆ alkyl, CF₃, N(C₁₋₆ alkyl)₂, Cl, F, Br, I, phenyl, benzyl, —CN, and NO₂;
R³ is selected from H, C₁₋₆ alkyl, OC₁₋₆ alkyl, CF₃, N(C₁I₆ alkyl)₂, Cl, F, Br, I, phenyl, benzyl, —CN, and NO₂;
R⁴ is selected from H, C₁₋₆ alkyl, OC₁₋₆ alkyl, CF₃, N(C₁₋₆ alkyl)₂, Cl, F, Br, I, phenyl, benzyl, —CN, and NO₂;
R⁵ is selected from C₁₋₂₀ alkyl, phenyl, and benzyl;
R⁶ is selected from C₁₋₈ alkyl, phenyl, and benzyl;
R⁷ is selected from C₁₋₁₈ alkyl, phenyl, and benzyl; and
R⁸ is selected from C₁₋₁₈ alkyl, phenyl, and benzyl.

2. A process of claim 1, wherein
(a) is performed in the presence of a first solvent;
the first solvent is capable of forming an azeotrope;
R¹ is selected from H, C₁₋₄ alkyl, Cl, F, and benzyl;
R² is selected from H, C₁₋₄ alkyl, Cl, F, and benzyl;
R³ is selected from H, C₁₋₄ alkyl, Cl, F, and benzyl;
R⁴ is selected from H, C₁₋₄ alkyl, Cl, F, phenyl, and benzyl;
R⁵ is selected from C₁₋₆ alkyl, phenyl, and benzyl;
R⁶ is selected from C₁₋₆ alkyl, phenyl, and benzyl;
R⁷ is selected from C₁₋₆ alkyl, phenyl, and benzyl; and
R⁸ is selected from C₁₋₆ alkyl, phenyl, and benzyl.

3. A process of claim 2, wherein
the first solvent is selected from toluene and benzene;
R¹ is selected from H and CH₃;
R² is selected from H and CH₃;
R³ is selected from H and CH₃;
R⁴ is selected from H and CH₃;
R⁵ is selected from C₁₋₆ alkyl;
R⁶ is selected from C₁₋₆ alkyl;
R⁷ is selected from C₁₋₆ alkyl; and
R⁸ is selected from C₁₋₆ alkyl.

4. A process of claim 1, wherein the first solvent is toluene;
R₁ is H;
R² is H;
R³ is H;
R⁴ is H;
R⁵ is n-butyl;
R⁶ is n-butyl;
R⁷ is n-butyl; and
R⁸ is n-butyl.

5. A process for preparing a compound of formula V: 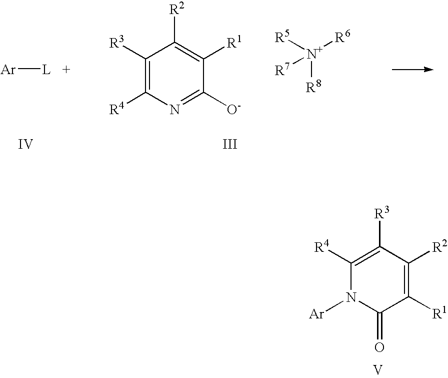
comprising:
(b) contacting a compound of formula IV with a compound of formula III in the presence of a metal salt and a second solvent; wherein:
metal salt is selected from a copper and a palladium salt;
the second solvent is an alcoholic or an aprotic solvent;
R¹ is selected from H, C₁₋₆ alkyl, OC₁₋₆ alkyl, CF₃, N(C₁₋₆ alkyl)₂, Cl, F, Br, I, phenyl, benzyl, —CN, and NO₂;
R² is selected from H, C₁₋₆ alkyl, OC₁₋₆ alkyl, CF₃, N(C₁₋₆ alkyl)₂, Cl, F, Br, I, phenyl, benzyl, —CN, and NO₂;
R³ is selected from H, C₁₋₆ alkyl, OC₁₋₆ alkyl, CF₃, N(C₁₋₆ alkyl)₂, Cl, F, Br, I, phenyl, benzyl, —CN, and NO₂;
R⁴ is selected from H, C₁₋₆ alkyl, OC₁₋₆ alkyl, CF₃, N(C₁₋₆ alkyl)₂, Cl, F, Br, I, phenyl, benzyl, —CN, and NO₂;
R⁵ is selected from C₁₋₂₀ alkyl, phenyl, and benzyl;
R⁶ is selected from C₁₋₈ alkyl, phenyl, and benzyl;
R⁷ is selected from C₁₋₁₈ alkyl, phenyl, and benzyl;
R⁸ is selected from C₁₋₁₈ alkyl, phenyl, and benzyl;
L is a leaving group;
Ar is an optionally substituted 5-10 membered aromatic carbocycle or heterocycle consisting of: carbon atoms and 04 heteroatoms selected from O, N, and S(O)ₚ; and
p is selected from 0, 1, and 2.

6. A process of claim 5, wherein the process, comprises: a process for preparing a compound of formula Va: 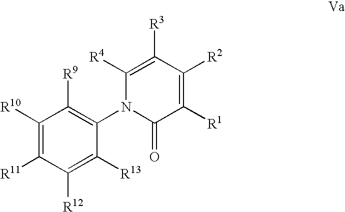
comprising:
(b) contacting a compound of formula IVa with a compound of formula III in the presence of a metal salt and a second solvent; 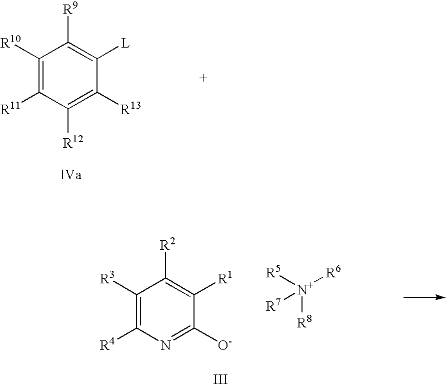
wherein:
the metal salt is a copper (I) salt;
the second solvent is an aprotic solvent;
R¹ is selected from H, C₁₋₄ alkyl, Cl, F, and benzyl;
R² is selected from H, C₁₋₄ alkyl, Cl, F, and benzyl;
R³ is selected from H, C₁₋₄ alkyl, Cl, F, and benzyl;
R⁴ is selected from H, C₁₋₄ alkyl, Cl, F, phenyl, and benzyl;
R⁵ is selected from C₁₋₆ alkyl, phenyl, and benzyl;
R⁶ is selected from C₁₋₆ alkyl, phenyl, and benzyl;
R⁷ is selected from C₁₋₆ alkyl, phenyl, and benzyl;
R⁸ is selected from C₁₋₆ alkyl, phenyl, and benzyl;
L is a leaving group selected from a halogen and a sulfonate;
R⁹ is selected from H, C₁₋₆ alkyl, Cl, and F;
R¹⁰ is selected from H, C₁₋₆ alkyl, phenyl, benzyl, C₁₋₆ alkyl-OH, O—C₁₋₆ alkyl, C(O)—C₁₋₆ alkyl, CO₂—C₁I₆ alkyl, C(O)NH₂, C(O)NH(C₁₋₄ alkyl), C(O)N(C₁₋₄ alkyl)₂, NHC(O)C₁₋₄ alkyl, N(C₁₋₄ alkyl)C(O)C₁₋₄ alkyl, S(O)ₚ—C₁₋₆ alkyl, S(O)ₚNH₂, S(O)ₚNH(C₁₋₄ alkyl), S(O)ₚN(C₁₋₄ alkyl)₂, NH₂, NH(C₁₋₄ alkyl), N(C₁₋₄ alkyl)₂, C₁₋₄ alkylene-NH₂, C₁₋₄ alkylene-NH(C₁₋₄ alkyl), C₁₋₄ alkylene-N(C₁₋₄ alkyl)₂, and NO₂;
R¹¹ is selected from H, C₁₋₆ alkyl, phenyl, benzyl, C₁₋₆ alkyl-OH, O—C₁₋₆ alkyl, C(O)—C₁₋₆ alkyl, CO₂—C₁₋₆ alkyl, C(O)NH₂, C(O)NH(C₁₋₄ alkyl), C(O)N(C₁₋₄ alkyl)₂, NHC(O)C₁₋₄ alkyl, N(C₁₋₄alkyl)C(O)C₁₋₄ alkyl, S(O)ₚ—C₁₋₆ alkyl, S(O)ₚNH₂, S(O)ₚNH(C₁₋₄ alkyl), S(O)ₚN(C₁₋₄ alkyl)₂, NH₂, NH(C₁₋₄ alkyl), N(C₁₋₄ alkyl)₂, C₁₋₄ alkylene-NH₂, C₁₋₄ alkylene-NH(C₁₋₄ alkyl), C₁₋₄ alkylene-N(C₁₋₄ alkyl)₂, and NO₂;
R¹² is selected from H, C₁₋₆ alkyl, phenyl, benzyl, C₁₋₆ alkyl-OH, O—C₁₋₆ alkyl, C(O)—C₁₋₆ alkyl, CO₂—C₁₋₆ alkyl, C(O)NH₂, C(O)NH(C₁₋₄ alkyl), C(O)N(C₁₋₄ alkyl)₂, NHC(O)C₁₋₄ alkyl, N(C₁₋₄alkyl)C(O)C₁₋₄ alkyl, S(O)ₚ—C₁₋₆ alkyl, S(O)ₚNH₂, S(O)ₚNH(C₁₋₄ alkyl), S(O)ₚN(C₁₋₄ alkyl)₂, NH₂, NH(C₁₋₄ alkyl), N(C₁₋₄ alkyl)₂, C₁₋₄ alkylene-NH₂, C₁₋₄ alkylene-NH(C₁₋₄ alkyl), C₁₋₄ alkylene-N(C₁₋₄ alkyl)₂, and NO₂;
R¹³ is selected from H, C₁₋₆ alkyl, Cl, and F; and
p, at each occurrence, is selected from 0, 1, and 2.

7. A process of claim 6, wherein
the metal salt is selected from CuI and CuOTf;
the second solvent is DMF;
R¹ is selected from H and CH₃;
R² is selected from H and CH₃;
R³ is selected from H and CH₃;
R⁴ is selected from H and CH₃;
R⁵ is selected from C₁₋₆ alkyl;
R⁶ is selected from C₁₋₆ alkyl;
R⁷ is selected from C₁₋₆ alkyl;
R⁸ is selected from C₁₋₆ alkyl;
L is a leaving group selected from Cl, Br, I, OSO₂Me, OSO₂CF₃, OSO₂Ph, and OSO₂Ph-p-Me;
R⁹ is selected from H, Cl₁₋₄ alkyl, Cl, and F;
R¹⁰ is selected from H, C₁₋₄ alkyl, phenyl, benzyl, O—C₁₋₄ alkyl, C(O)—C₁₋₄ alkyl, CO₂—C₁₋₄ alkyl, and NO₂;
R¹¹ is selected from H, C₁₋₄ alkyl, phenyl, benzyl, O—C₁₋₄ alkyl, C(O)—C₁₋₄ alkyl, CO₂—C₁₋₄ alkyl, NH₂, and NO₂;
R¹² is selected from H, C₁₋₄ alkyl, phenyl, benzyl, O—C₁₋₄ alkyl, C(O)—C₁₋₄ alkyl, CO₂—C₁₋₄ alkyl, and NO₂; and
R¹³ is selected from H, C₁₋₄ alkyl, Cl, and F.

8. A process of claim 7, wherein
the metal salt is CuI;
the second solvent is DMF;
R¹ is H;
R² is H;
R³ is H;
R⁴ is H;
R⁵ is n-butyl;
R⁶ is n-butyl;
R⁷ is n-butyl;
R⁸ is n-butyl;
L is I;
R⁹ is H;
R¹⁰ is selected from H, CH₃, CH₂CH₃, CH(CH₃)₂, and NO₂;
R¹ I is selected from H, C₁₋₄ alkyl, OCH₃, CO₂CH₂CH₃, NH₂, and NO₂;
R¹² is selected from H, C₁₋₄ alkyl, and NO₂; and
R¹³ is H.

9. A compound of formula III: 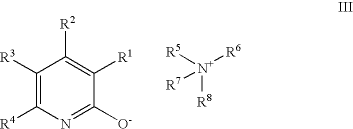
wherein:
R¹ is selected from H, C₁₋₆ alkyl, OC₁₋₆ alkyl, CF₃, N(C₁₋₆ alkyl)₂, Cl, F, Br, I, phenyl, benzyl, —CN, and NO₂;
R² is selected from H, C₁₋₆ alkyl, OC₁₋₆ alkyl, CF₃, N(C₁₋₆ alkyl)₂, Cl, F, Br, I, phenyl, benzyl, —CN, and NO₂;
R³ is selected from H, C₁₋₆ alkyl, OC₁₋₆ alkyl, CF₃, N(C₁₋₆ alkyl)₂, Cl, F, Br, I, phenyl, benzyl, —CN, and NO₂;
R⁴ is selected from H, C₁₋₆ alkyl, OC₁₋₆ alkyl, CF₃, N(C₁₋₆ alkyl)₂, Cl, F, Br, I, phenyl, benzyl, —CN, and NO₂;
R⁵ is selected from C₁₋₂₀ alkyl, phenyl, and benzyl;
R⁶ is selected from C₁₋₁₈ alkyl, phenyl, and benzyl;
R⁷ is selected from C₁₋₁₈ alkyl, phenyl, and benzyl; and
R⁸ is selected from C-₁₋₁₈ alkyl, phenyl, and benzyl.

10. A compound of formula Va: 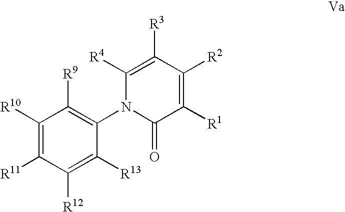
wherein:
R¹ is selected from H, C₁₋₄ alkyl, Cl, F, and benzyl;
R² is selected from H, C₁₋₄ alkyl, Cl, F, and benzyl;
R³ is selected from H, C₁₋₄ alkyl, Cl, F, and benzyl;
R⁴ is selected from H, C₁₋₄ alkyl, Cl, F, phenyl, and benzyl;
R⁹ is selected from H, C₁₋₆ alkyl, Cl, and F;
R¹⁰ is selected from H, C₁₋₆ alkyl, phenyl, benzyl, C₁₋₆ alkyl-OH, O—C₁₋₆ alkyl, C(O)—C₁I₆ alkyl, CO₂—C₁₋₁₆ alkyl, C(O)NH₂, C(O)NH(C₁₋₄ alkyl), C(O)N(C₁₋₄ alkyl)₂, NHC(O)C₁₋₄ alkyl, N(C₁₋₄ alkyl)C(O)C₁₋₄ alkyl, S(O)ₚ—C₁₋₆ alkyl, S(O)ₚNH₂, S(O)ₚNH(C₁₋₄ alkyl), S(O)ₚN(C₁₋₄ alkyl)₂, NH₂, NH(C₁₋₄ alkyl), N(C₁₋₄ alkyl)₂, C₁₋₄ alkylene-NH₂, C₁₋₄ alkylene-NH(C₁₋₄ alkyl), C₁₋₄ alkylene-N(C₁₋₄ alkyl)₂, and NO₂;
R¹¹ is selected from H, C₁₋₆ alkyl, phenyl, benzyl, C₁₋₆ alkyl-OH, O—C₁₋₆ alkyl, C(O)—C₁₋₆ alkyl, CO₂—C₁₋₆ alkyl, C(O)NH₂, C(O)NH(C₁₋₄ alkyl), C(O)N(C₁₋₄ alkyl)₂, NHC(O)C₁₋₄ alkyl, N(C₁₋₄alkyl)C(O)C₁₋₄ alkyl, S(O)ₚ—C₁₋₆ alkyl, S(O)ₚNH₂, S(O)ₚNH(C₁₋₄ alkyl), S(O)ₚN(C₁₋₄ alkyl)₂, NH₂, NH(C₁₋₄ alkyl), N(C₁₋₄ alkyl)₂, C₁₋₄ alkylene-NH₂, C₁₋₄ alkylene-NH(C₁₋₄ alkyl), C₁₋₄ alkylene-N(C₁₋₄ alkyl)₂, and NO₂;
R¹² is selected from H, C₁₋₆ alkyl, phenyl, benzyl, C₁₋₆ alkyl-OH, O—C₁₋₆ alkyl, C(O)—C₁₋₆ alkyl, CO₂—C₁₋₆ alkyl, C(O)NH₂, C(O)NH(C₁₋₄ alkyl), C(O)N(C₁₋₄ alkyl)₂, NHC(O)C₁₋₄ alkyl, N(C₁₋₄ alkyl)C(O)C₁₋₄ alkyl, S(O)ₚ—C₁₋₆ alkyl, S(O)ₚNH₂, S(O)ₚNH(C₁₋₄ alkyl), S(O)ₚN(C₁₋₄ alkyl)₂, NH₂, NH(C₁₋₄ alkyl), N(C₁₋₄ alkyl)₂, C₁₋₄ alkylene-NH₂, C₁₋₄ alkylene-NH(C₁₋₄ alkyl), C₁₋₄ alkylene-N(C₁₋₄ alkyl)₂, and NO₂;
R¹³ is selected from H, C₁₋₆ alkyl, Cl, and F; and
p, at each occurrence, is selected from 0, 1, and 2.
